(19) United States
(12) Patent Application Publication
(10) Pub. No.: US 2013/0345668 A1
(43) Pub. Date: Dec. 26, 2013

(54) SKIN SECURABLE INFUSION ASSEMBLY AND METHOD OF USE

(71) Applicant: Cook Medical Technologies LLC, Bloomington, IN (US)

(72) Inventor: Arman Valaie, Bloomington, IN (US)

(73) Assignee: Cook Medical Technologies LLC, Bloomington, IN (US)

(21) Appl. No.: 13/916,654

(22) Filed: Jun. 13, 2013

Related U.S. Application Data
(60) Provisional application No. 61/661,987, filed on Jun. 20, 2012.

(51) Int. Cl. A61M 5/142; A61M 5/145

(52) U.S. Cl. CPC A61M 5/14248; A61M 5/1456
USPC 604/506; 604/152

(57) ABSTRACT

A skin securable infusion assembly includes a housing having a skin contacting adhesive layer and supporting an infusion cannula. An elongate tubular reservoir having a plunger slidably disposed therein is configured to contain a supply of liquid medication between a distal opening of the reservoir and the plunger. The reservoir has an assembled configuration in which the reservoir is supported on the housing. An actuation mechanism is coupled with the plunger and, when the reservoir is in the assembled configuration, an actuation of the actuation mechanism moves the plunger toward the distal opening and forces a portion of the liquid medication through the distal opening and into the infusion cannula.

### RELATION TO OTHER PATENT APPLICATION

This application claims priority to provisional patent application 61/661,987, filed on Jun. 20, 2012, with the same title.

### TECHNICAL FIELD

The present disclosure relates generally to a skin securable infusion assembly, and more particularly to an elongate tubular reservoir supported on a housing of the assembly and having a plunger disposed therein that is movable by an actuation mechanism to administer liquid medication.

### BACKGROUND

Insulin may be administered in a number of different ways. For example, insulin may be administered using vial drawn injections, pre-filled cartridges or devices, indwelling catheters, and even subcutaneous infusion pumps. Both long acting insulin and short acting insulin may be required and dosage amounts may vary on a daily basis, depending on the specific patient needs. Infusion pump systems typically include an electronic pump, which may be carried or worn by the patient, that delivers required insulin to the patient from a reservoir carried on the pump through a catheter and infusion set. The electronic pump generally includes a control program for administering continuous and/or discrete dosages of insulin to the patient. Some electronic pumps also include or interact with devices for measuring blood glucose levels of the patient and adjust the amount of insulin administered to the patient based on the detected glucose levels.

Although insulin pump systems may provide a high level of control over blood glucose levels, the pump systems are relatively costly and complex. For example, some of the pump systems require significant programming input by the patient using the pump system in order for the electronic pump to provide the sophisticated electronic control. In addition, because the electronic pump delivers insulin from a reservoir carried within the electronic pump, the infusion set and catheter must be primed with insulin. Thus, if the reservoir or other component of the pump requires replacement, or if the catheter and/or infusion set become disconnected from the pump and reservoir, the amount of insulin primed within the catheter and infusion set may be wasted. The pump systems may also be undesirable for patients that do not wish to wear or carry a plurality of components.

The present disclosure is directed toward one or more of the problems set forth above.

### SUMMARY OF THE DISCLOSURE

In one aspect, a skin securable infusion assembly includes a housing having a skin contacting adhesive layer and supporting an infusion cannula. An elongate tubular reservoir having a plunger slidably disposed therein is configured to contain a supply of liquid medication between a distal opening of the reservoir and the plunger. The reservoir has an assembled configuration in which the reservoir is supported on the housing. An actuation mechanism is coupled with the plunger and, when the reservoir is in the assembled configuration, an actuation of the actuation mechanism moves the plunger toward the distal opening and forces a portion of the liquid medication through the distal opening and into the infusion cannula.

In another aspect, a method of administering a liquid medication using a skin securable infusion assembly includes a step of adhering an adhesive layer of a housing of the skin securable infusion assembly to skin of a patient. An infusion cannula supported on the housing is inserted beneath the skin. A discrete dosage of the liquid medication is administered to the patient by moving a plunger disposed within a reservoir of the skin securable infusion assembly toward a distal opening of the reservoir using an actuation mechanism coupled with the plunger. This movement of the plunger forces the discrete dosage of the liquid medication through the distal opening and into the infusion cannula.

In yet another aspect, a disposable reservoir for a skin securable infusion assembly includes an elongate tubular reservoir having a spiral configuration. A distal opening of the reservoir is adapted for mating engagement with an infusion cannula of a skin securable infusion assembly housing, and a proximal opening of the reservoir is adapted for mating engagement with an actuation mechanism. A plunger is slidably disposed within the reservoir and movable in response to actuation of the actuation mechanism. A supply of liquid medication is contained within the reservoir between the distal opening of the reservoir and the plunger.

### BRIEF DESCRIPTION OF THE DRAWINGS

FIG. 1 is a top perspective view of a skin securable infusion assembly, according to one embodiment of the present disclosure;

FIG. 2 is a bottom perspective view of the skin securable infusion assembly of FIG. 1;

FIG. 3 is a bottom perspective view of one embodiment of an actuation mechanism for use with the skin securable infusion assembly of FIGS. 1 and 2, according to one aspect of the present disclosure;

FIG. 4 is a diagrammatic view of the actuation mechanism of FIG. 3, shown in combination with the elongate tubular reservoir of the skin securable infusion assembly of FIGS. 1 and 2, according to another aspect of the present disclosure; and

FIG. 5 is a diagrammatic view of another exemplary embodiment of an actuation mechanism for use with the skin securable infusion assembly of FIGS. 1 and 2, according to another aspect of the present disclosure.

### DETAILED DESCRIPTION

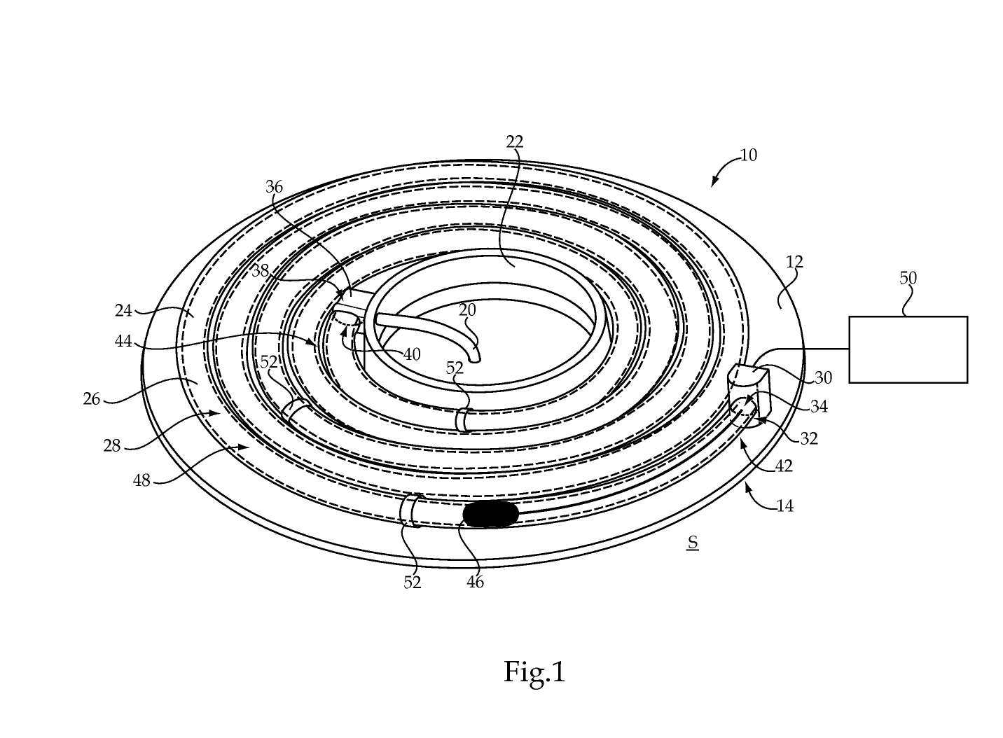

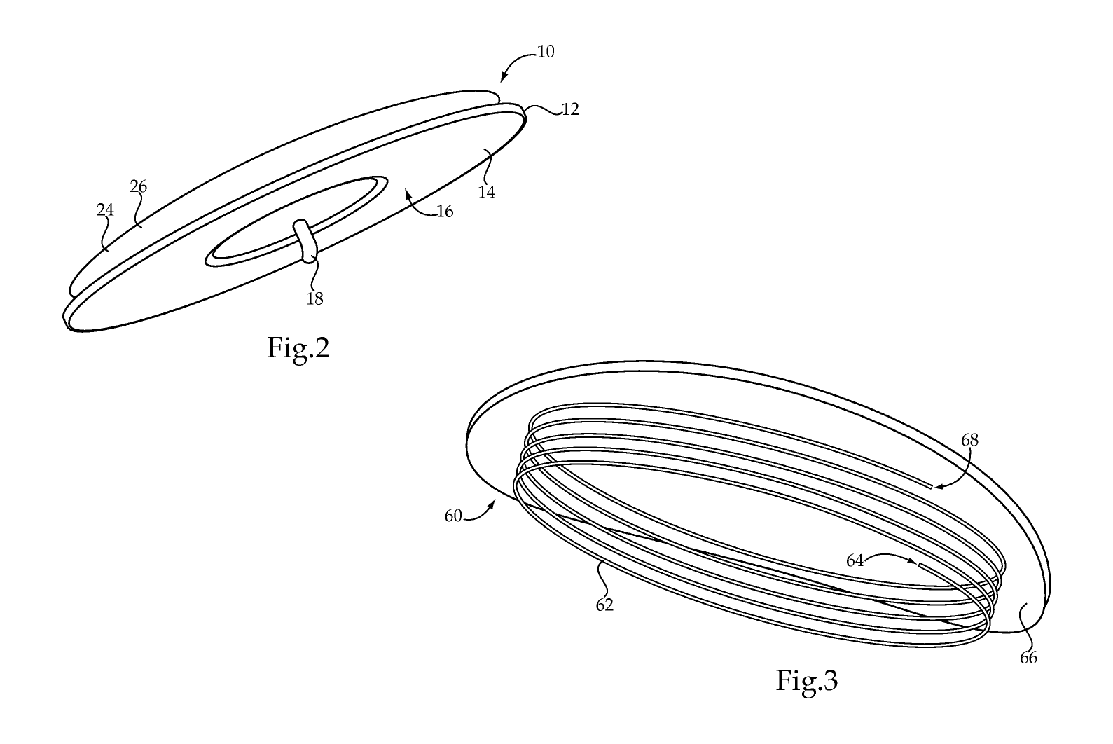

Referring to FIG. 1, there is shown a skin securable infusion assembly 10 according to the present disclosure. The skin securable infusion assembly 10 may generally include a housing 12, such as a cylindrical housing, having a skin contacting adhesive layer 14. Referring also to FIG. 2, the skin contacting adhesive layer 14 may, for example, define a bottom surface 16 of the housing 12 and may include a medical grade adhesive tape, microbarbs, or other known adhesive means for securing the skin securable infusion assembly 10 to skin S of a patient. The housing 12 may also support an infusion cannula 18, which may be inserted beneath the skin S using a needle or other insertion device. The subcutaneous cannula 18 may include or may be integral with a dispensing catheter 20, as shown. According to the exemplary embodiment, the housing 12 may include a supporting structure 22 receiving a portion of the dispensing catheter 20 therethrough.

The skin securable infusion assembly 10 may also include an elongate tubular reservoir 24 having tubular walls 26 defining a lumen 28 for containing a liquid medication, such as insulin. The reservoir 24 may have an outer diameter that is orders of magnitude smaller than its length and, according to some embodiments, may be provided in a spiral configuration, as shown. The reservoir 24 is also shown in an assembled configuration in which the reservoir 24 is supported on the housing 12. A proximal pressure valve 30 may be positioned over a proximal opening 32 of the reservoir 24 and may include a proximal valve member 34 movable between open and closed positions. A distal pressure valve 36 may be positioned over a distal opening 38 of the reservoir 24 and may include a distal valve member 40 movable between open and closed positions. As shown, the distal pressure valve 36 may also support the dispensing catheter 20 such that when the distal valve member 40 moves to an open position the reservoir 24 is fluidly connected with the dispensing catheter 20 and, thus, the infusion cannula 18.

The pressure valves 30 and 36 may be fixedly supported on the housing 12 and may provide a means for securing the reservoir 24 to the housing 12. For example, to support the reservoir 24 on the housing 12 in the assembled configuration introduced above, a proximal end 42 of the reservoir 24 may be inserted into or connected with the proximal pressure valve 30, while a distal end 44 of the reservoir 24 may be inserted into or connected with the distal pressure valve 36. Alternatively, the pressure valves 30 and 36 may be provided on the respective ends 42 and 44 of the reservoir 24 before the reservoir is secured to the housing 12. As such, the reservoir and/or the pressure valves 30 and 36 may be secured to the housing using known means, such as, for example, adhesives, clips, clamps, and the like. Alternatively, according to embodiments not utilizing pressure valves 30 and 36, the tubular walls 26 of the reservoir 24 may alone be secured to the housing 12.

A plunger 46, which may, for example, include a floating silicon cylinder, may be slidably disposed within the elongate tubular reservoir 24. A supply of liquid medication 48 may be contained in the reservoir 24 between the plunger 46 and the distal opening 38 of the reservoir 24. The reservoir 24 may be pre-filled with the supply of liquid medication 48 and, thus, the plunger 46 may be initially positioned in close proximity to the proximal opening 32. Alternatively, the reservoir 24 may be assembled with the housing 12 in an empty state, requiring the liquid medication 48 to be loaded into the reservoir 24 prior to use. Although known methods exist for loading liquid medication 48 into the reservoir 24, it should be appreciated that some methods may include moving the plunger 46 in the distal to proximal direction to reduce pressure within the reservoir 24 and draw in the medication 48. Of course, syringes or other devices may be used to push the liquid medication 48 into the reservoir 24.

As will be discussed later in greater detail, the skin securable infusion assembly 10 may also include an actuation mechanism, shown generally at block 50, coupled with the plunger 46. Although particular embodiments may vary, actuation of the actuation mechanism 50 will preferably move the plunger 46 toward the distal opening 38 of the reservoir 24 and, thus, force a portion of the liquid medication 48 through the distal opening 38 and into the infusion cannula 18. As shown, the reservoir 24 may be transparent, such that a location of the plunger 46 within the reservoir 24 is visible to provide an indication of a remaining volume of the supply of liquid medication 48 within the reservoir 24. According to some embodiments, markings 52 may be provided at discrete locations along the length of the reservoir 24 and may correspond to specific volumes in order to provide a precise indication of the remaining amount of liquid medication 48.

Turning now to FIG. 3, a first exemplary embodiment of an actuation mechanism 60 may include a flexible mandrel 62 having a distal end 64 configured to contact the plunger 46. The mandrel 62 may be flexible to follow the curvature of the reservoir 24 during advancement, but firm enough to move the plunger 46 toward the distal opening 38 of the reservoir 24 during the advancement. According to some embodiments, the mandrel 62 may be provided in a spiral, or coiled, configuration. As shown, the actuation mechanism 60 may also include a cap 66 supported on the housing 12 and rotatable with respect to the housing 12 for moving the mandrel 62. For example, a proximal end 68 of the mandrel 62 may be fixedly attached to the cap 66, while the distal end 64 of the mandrel 62 may be supported for telescopic insertion into the reservoir 24. As should be appreciated, a length of the mandrel 62 may substantially correspond to a length of the reservoir 24. The cap 66 may include any known attachment means, including clips, clamps, and the like, and may further include known means for allowing rotation of the cap 66 relative to the housing 12. The cap 66 and/or housing 12 may include a curved or rounded profile to reduce friction with garments and the like.

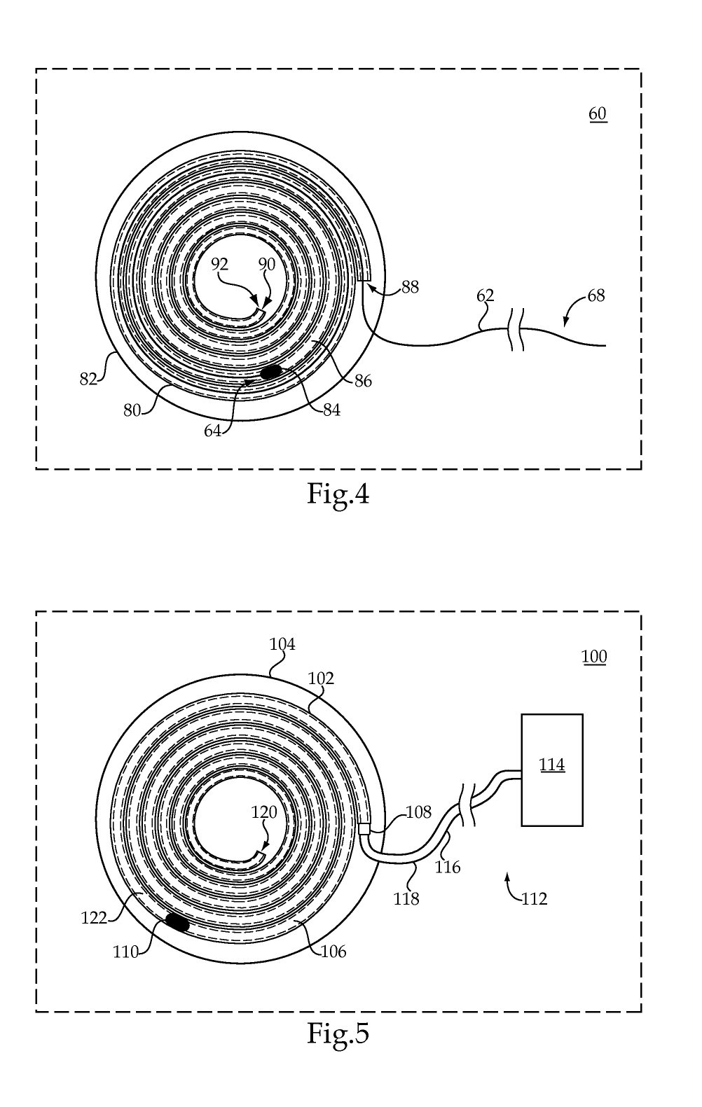

Referring also to FIG. 4, the mandrel 62 is shown telescopically received within an exemplary reservoir 80 supported on a housing 82. In particular, a plunger 84 disposed within the reservoir 80 has been moved toward a distal opening 86 of the reservoir 80 using the distal end 64 of the mandrel 62. The distal end 64 of the mandrel 62 may be attached to or in contact with the plunger 84 and may be telescopically received within the reservoir a distance corresponding to a rotation amount of the cap 66. Markings may be provided on the cap 66 and/or housing 82 to assist in administering precise dosages of a liquid medication 86. Further, the cap 66 and housing 82 may interface such that the cap 66 is permitted to rotate at discrete amounts, with one discrete rotation amount, or click, corresponding to a specific unit or amount of the liquid medication 86. As a result, the patient may administer the appropriate amount of liquid medication 86 by rotating the cap 66 one, two, three, or more discrete rotation amounts, or clicks.

According to the particular embodiment, the reservoir 80 may be provided without the pressure valves described above. In particular, the plunger 84 may prevent the liquid medication 86 from leaking through a proximal opening 88 of the reservoir, while a small orifice 90 provided at a distal end 92 of the reservoir 80, along with reduced pressure in the reservoir 80, may sufficiently prevent leakage at the distal end 92 unless and until the plunger 84 is advanced using the mandrel 62 to force a portion of the liquid medication 86 through the orifice 90. Yet alternatively, the small orifice 90 may be unnecessary and the distal end 92 may include a conventional opening that is fluidly connected with a dispensing catheter and cannula, such as the dispensing catheter 20 and cannula 18 discussed above. As such, the cannula 18 may sufficiently reduce leakage until the plunger 84 is actuated.

According to an alternative actuation mechanism 100, and as shown in FIG. 5, a reservoir 102, supported on a housing 104, may contain a supply of pressurized fluid 106 between a proximal pressure valve 108 and a plunger 110. For example, a supply of liquid or air may be provided between the proximal pressure valve 108 and the plunger 110 for movement of the plunger 110 within the reservoir 102. A pump assembly 112, which may include a pump 114, such as an electronic pump, and a connecting catheter 116, may be provided for pumping an additional amount of fluid 118 into the reservoir 102 through the proximal pressure valve 108. Upon actuation of the pump assembly 112, the additional amount of fluid 118 moves the plunger 110 toward a distal end 120 of the reservoir 102 to force a portion of liquid medication 122, contained within the reservoir 102 between the plunger 110 and the distal end 120, from the reservoir 122 for administration to the patient.

### INDUSTRIAL APPLICABILITY

The present disclosure is generally applicable to skin securable, or wearable, infusion assemblies. More specifically, the present disclosure finds application with skin securable infusion assemblies having an elongate tubular reservoir supported on a housing of the assembly and including a plunger movable therein by an actuation mechanism. Yet further, the present disclosure finds application in relatively compact skin securable infusion assemblies for self administering discrete dosages of insulin.

Referring generally to FIGS. 1-5, a skin securable infusion assembly 10 may be provided for administering discrete dosages of a liquid medication, such as insulin. Referring specifically to FIGS. 1 and 2, the skin securable infusion assembly 10 may generally include a housing 12 having a skin contacting adhesive layer 14 and supporting an infusion cannula 18. An elongate tubular reservoir 24, shown in a spiral configuration, may be provided in an attached configuration in which the reservoir 24 is supported on the housing 12. In the attached configuration, a distal end 44, or distal opening 38, of the reservoir 24 may interface with a dispensing catheter 20 and/or the infusion cannula 18. A plunger 46 is slidably disposed within the reservoir 24, with a supply of liquid medication 48 being contained within the reservoir 24 between the plunger 46 and the distal opening 38. An actuation mechanism 50 is coupled with the plunger 46 to advance the plunger 46 and dispense a portion of the liquid medication 48.

The skin securable infusion assembly 10 may be used by a patient to self-administer discrete dosages of a liquid medication, such as insulin. For example, at an initial stage, the skin securable infusion assembly 10 may be secured to the skin S of the patient by adhering the skin contacting adhesive layer 14 of the housing 12 to the skin S. The infusion cannula 18 may be inserted beneath the skin S using a needle or insertion device, as is know by those skilled in the art. A discrete dosage of the liquid medication 48 may be then administered, as needed, to the patient by moving the plunger 46 toward the distal opening 38 using the actuation mechanism 50 and forcing the discrete dosage of the liquid medication 48 through the distal opening 38 and into the infusion cannula 18.

According to one exemplary embodiment, shown in FIGS. 3 and 4, a cap 66 supported on the housing 12 may be rotated with respect to the housing 12 to advance a distal end 64 of a flexible mandrel 62 within a reservoir 80. In particular, the distal end 64 may contact or be connected with a plunger 84, such that telescopic advancement of the mandrel 62 with respect to the reservoir 80 pushes the plunger 84 toward a distal opening 86 of the reservoir 80. As such, a dosage of the medication 86 corresponding to the actuation amount, or rotation of the cap 66, is dispensed from the reservoir 80.

Alternatively, and referring to FIG. 5, a plunger 110 may be moved using pressurized fluid. In particular, a supply of pressurized fluid 106 may be provided between a proximal pressure valve 108 and the plunger 110. A pump assembly 112, which may include a pump 114 and a connecting catheter 116, may be provided for pumping an additional amount of fluid 118 into the reservoir 102 through the proximal pressure valve 108. Specifically, upon actuation of the pump assembly 112, the additional amount of fluid 118 moves the plunger 110 toward a distal end 120 of the reservoir 102 to force a portion of liquid medication 122, corresponding to a discrete actuation of the pump 114, from the reservoir 122 for administration to the patient.

The skin securable infusion assembly disclosed herein provides a compact and convenient means for self-administering discrete dosages of insulin. Due to the close proximity of the reservoir to the dispensing catheter and/or infusion cannula, a long priming catheter, which may at times result in wasted insulin, is not required. According to some embodiments, the assembly may be self-contained, while other embodiments may incorporate the use of a pump, which may include, for example, the conventional electronic pumps. According to other embodiments, a patient has the flexibility to switch between the alternative actuation mechanisms, depending on daily requirements and/or preferences. The reservoir and/or housing may be disposable, while the actuation mechanism may be reusable, depending on the particular embodiment. As such, the infusion assembly disclosed herein may be provided as a less costly alternative to conventional wearable infusion systems.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A skin securable infusion assembly, comprising:
a housing having a skin contacting adhesive layer and supporting an infusion cannula;
an elongate tubular reservoir having a plunger slidably disposed therein, wherein the reservoir is configured to contain a supply of liquid medication between a distal opening of the reservoir and the plunger, wherein the reservoir has an assembled configuration in which the reservoir is supported on the housing; and
an actuation mechanism coupled with the plunger, wherein, in the assembled configuration, an actuation of the actuation mechanism moves the plunger toward the distal opening and forces a portion of the liquid medication through the distal opening and into the infusion cannula.

2. The skin securable infusion assembly of claim 1, wherein, in the assembled configuration, the reservoir has a spiral configuration.

3. The skin securable infusion assembly of claim 2, wherein the reservoir is transparent.

4. The skin securable infusion assembly of claim 3, wherein the reservoir includes a marking thereon corresponding to a remaining volume of the supply of liquid medication.

5. The skin securable infusion assembly of claim 2, wherein the actuation mechanism includes a flexible mandrel having a distal end contacting the plunger, wherein the flexible mandrel is telescopically advanced within the reservoir responsive to the actuation.

6. The skin securable infusion assembly of claim 5, wherein the actuation mechanism further includes a cap supported on the housing and rotatable with respect to the housing for moving the mandrel responsive to the actuation.

7. The skin securable infusion assembly of claim 2, further including a distal pressure valve positioned over the distal opening and having a distal valve member movable between open and closed positions.

8. The skin securable infusion assembly of claim 7, further including a proximal pressure valve positioned over a proximal opening of the reservoir and having a proximal valve member movable between open and closed positions.

9. The skin securable infusion assembly of claim 8, wherein the reservoir contains a supply of pressurized fluid between the proximal pressure valve and the plunger.

10. The skin securable infusion assembly of claim 9, wherein the actuation mechanism includes a pump assembly for pumping additional pressurized fluid into the reservoir through the proximal pressure valve.

11. A method of administering a liquid medication using a skin securable infusion assembly, wherein the skin securable infusion assembly includes a housing having a skin contacting adhesive layer and supporting an infusion cannula, an elongate tubular reservoir having a plunger slidably disposed therein, wherein the reservoir is supported on the housing and contains a supply of liquid medication between a distal opening of the reservoir and the plunger, and an actuation mechanism coupled with the plunger, the method comprising steps of:
adhering the adhesive layer of the housing to skin of a patient;
inserting the infusion cannula beneath the skin; and
administering a discrete dosage of the liquid medication to the patient by moving the plunger toward the distal opening using an actuation mechanism coupled with the plunger and forcing the discrete dosage of the liquid medication through the distal opening and into the infusion cannula.

12. The method of claim 11, further including supporting the reservoir on the housing in a spiral configuration.

13. The method of claim 12, wherein the administering step includes moving the plunger by contacting the plunger with a distal end of a flexible mandrel and telescopically advancing the flexible mandrel within the reservoir.

14. The method of claim 13, wherein the administering step further includes rotating a cap supported on the housing with respect to the housing to advance the flexible mandrel.

15. The method of claim 12, wherein the administering step includes moving a distal valve member of a distal pressure valve positioned over the distal opening from a closed position to an open position.

16. The method of claim 15, wherein the administering step further includes moving a proximal valve member of a proximal pressure valve positioned over a proximal opening of the reservoir from a closed position to an open position.

17. The method of claim 16, wherein the administering step further includes moving the plunger using a supply of pressurized fluid contained between the proximal pressure valve and the plunger.

18. The method of claim 17, wherein the administering step further includes pumping additional pressurized fluid into the reservoir through the proximal pressure valve using a pump assembly.

19. A disposable reservoir for a skin securable infusion assembly, wherein the skin securable infusion assembly includes a housing having a skin contacting adhesive layer and supporting an infusion cannula, the disposable reservoir comprising:
an elongate tubular reservoir having a spiral configuration, wherein a distal opening of the reservoir is adapted for mating engagement with the infusion cannula and a proximal opening of the reservoir is adapted for mating engagement with an actuation mechanism;
a plunger slidably disposed within the reservoir and movable in response to actuation of the actuation mechanism; and
a supply of liquid medication contained within the reservoir between the distal opening of the reservoir and the plunger.

20. The disposable reservoir of claim 19, wherein the reservoir is transparent and includes a marking thereon corresponding to a remaining volume of the supply of liquid medication.
